(19) United States
(12) Patent Application Publication
(10) Pub. No.: US 2011/0189697 A1
(43) Pub. Date: Aug. 4, 2011

(54) RECOMBINANT CELLS AND PLANTS FOR SYNTHESIS OF VERY LONG CHAINS FATTY ACID (VLCFA)

(75) Inventors: Jean- Denis Faure, Gif S/Yvette (FR); Liën Bach, Gif S/Yvette (FR); Martine Miquel, Elancourt (FR); Johnathan Napier, Hertfordshire (GB)

(73) Assignees: INSTITUTE NATIONAL DE LA RECHERCHE AGRONIMIQUE, Paris (FR); CENTRE NATIONAL DE LA RECHERCHE SCIENTIFIQUE (CNRS, Paris (FR); ROTHAMSTED RESEARCH LTD., Harpenden, Hertfordshire (GB)

(21) Appl. No.: 13/000,451

(22) PCT Filed: Jun. 25, 2009
(86) PCT No.: PCT/EP2009/057964

§ 371 Date: Apr. 15, 2011

(30) Foreign Application Priority Data
Jun. 27, 2008 (EP) 08159181.0

(51) Int. Cl. G01N 33/573; C12P 7/64

(52) U.S. Cl. 435/7.4; 435/134

(57) ABSTRACT

The present disclosure is relative to a method for the production of Very-Long-Chain Fatty Acids (VLCFA) into a plant cell, including culturing a recombinant plant cell in an appropriate medium, wherein said plant cell is transformed with an heterologous gene encoding for an hydroxyacyl-CoA dehydratase. The disclosure is also relative to a method for producing vegetable oil including high levels of VLCFA.

### CROSS-REFERENCE TO RELATED APPLICATIONS

This application is a National Phase Entry of International Application No. PCT/EP2009/057964, filed on Jun. 25, 2009, which claims priority to European Patent Office Application No. 08159181.0, filed on Jun. 27, 2008, both of which are incorporated by reference herein.

### INTRODUCTION

Living organisms synthesize a vast array of different fatty acids which are incorporated into complex lipids. These complex lipids represent both major structural component membranes, and are a major storage product in both plants and animals.

Very-long-chain fatty acids (VLCFAs) are components of eukaryotic cells and are composed of 20 or more carbons in length (i.e. >C18). VLCFAs are involved in many different physiological functions in different organisms. They are abundant constituents of some tissues like the brain (myelin) or plant seed (storage triacylglycerols, TAGs). VLCFAs are components of the lipid barrier of the skin and the plant cuticular waxes. The long acyl chain of certain VLCFAs is necessary for the high membrane curvature, found for instance in the nuclear pore. VLCFAs are also involved in the secretory pathway for protein trafficking and for the synthesis of GPI lipid anchor. Finally, VLCFAs are components of sphingolipids that are both membrane constituents and signalling molecules.

Very long chain fatty acids are synthesized in the epidermal cells where they are either directly incorporated into waxes, or serve as precursors for other aliphatic hydrocarbons found in waxes, including alkanes, primary and secondary alcohols, ketones aldehydes and acyl- esters. VLCFAs also accumulate in the seed oil of some plant species, where they are incorporated into triacylglycerols (TAGs), as in the Brassicaceae, or into wax esters, as in jojoba. These seed VLCFAs include the agronomically important erucic acid (C22: 1), used in the production of lubricants, nylon, cosmetics, pharmaceuticals and plasticizers.

In yeast and mammals, VLCFA synthesis is catalyzed in the Endoplasmic Reticulum by a membrane-bound multi-enzyme protein complex referred as the elongase. The elongase complex catalyzes the cyclic addition of a C2-moiety obtained from malonyl-Coenzyme A to an acyl-CoA. VLCFAs (C20, C22, C24 or higher) are produced from shorter fatty acids (usually C16 or C18) made by the cytolosic Fatty Acid Synthase complex (FAS). The two-carbon addition during the elongation cycle requires four independent but sequential enzymatic steps.

The first step involves the condensation of the malonyl-CoA with an acylₙ-CoA precursor resulting in 3-ketoacyl-CoA intermediate that is reduced to form a 3-hydroxy-acyl-CoA. The third enzymatic step is the dehydration of the 3-hydroxy-acyl-CoA to an enoyl-CoA that is finally reduced to yield an acylₙ₊₂-CoA. The component members of the elongase were recently fully described in yeast.

In plants, there is a large family of 3-ketoacyl-CoA synthases (KCS) condensing enzymes exemplified by the Arabidopsis gene Fatty Acid Elongase 1 (FAE1), required in seeds for the synthesis of the C20+ fatty acids such as erucic acid. The Arabidopsis genome encodes 21 FAE-like KCSs and although these enzymes are structurally unrelated to the ELO class of condensing enzymes, it has been demonstrated that several Arabidopsis FAE-KCSs can rescue the otherwise lethal yeast elo2Δ/elo3Δ double mutant. Below is presented a list of genes from Arabidopsis thaliana, encoding for enzymes belonging to the elongase complex:

| | Names found | | References |
|---|---|---|---|
| Gene names | in papers | Accession | (see below) |
| KCS1 | KCS1 | At1g01120 | 1, 2, 20 |
| KCS2 | | At1g04220 | 16, 21 |
| KCS3 | | At1g07720 | 1 |
| KCS4 | | At1g19440 | 1 |
| KCS5 | CER60 | At1g25450 | 2, 4, 21 |
| KCS6 | CER6, CUT1 | At1g68530 | 2, 4, 8, 11, 15, 21 |
| KCS7 | | At1g71160 | 1 |
| KCS8 | | At2g15090 | |
| KCS9 | | At2g16280 | 16 |
| KCS10 | FDH | At2g26250 | 2, 3, 13, 17, 19, 21, 22 |
| KCS11 | | At2g26640 | 1 |
| KCS12 | | At2g28630 | |
| KCS13 | HIC | At2g46720 | 2, 7 |
| KCS14 | | At3g10280 | |
| KCS15 | | At3g52160 | |
| KCS16 | | At4g34250 | 1 |
| KCS17 | | At4g34510 | 1, 12, 21 |
| KCS18 | KCS2 | At4g34520 | |
| KCS19 | FAE1 | At5g04530 | 1, 16 |
| KCS20 | | At5g43760 | 16, 21 |
| KCS21 | | At5g49070 | |
| KCR | | At1g67730 | 23 |
| ECR | CER10 | At3g55360 | 24, 25 |

References:
1, Blacklock and Jaworski (2006);
2, Costaglioli et al. (2005);
3, Efremova et al. (2004);
4, Fiebig et al. (2000);
5, Ghanevati and Jaworski (2001);
6, Ghanevati and Jaworski (2002);
7, Gray et al. (2000);
8, Hooker et al. (2002);
9, James and Dooner (1990);
10, James et al. (1995);
11, Kunst et al. (2000);
12, Kunst and Clemens (2001);
13, Lolle et al. (1997);
14, Millar and Kunst (1997);
15, Millar et al. (1999);
16, Paul et al. (2006);
17, Pruitt et al. (2000);
18, Rossak et al. (2001);
19, Stasolla et al. (2003);
20, Todd et al. (1999);
21, Trenkamp et al. (2004);
22, Yephremov et al. (1999);
23, Beaudoin et al. (2002);
24, Gable et al. (2004);
25, Zheng et al. (2005).

### BACKGROUND

The goal of the present invention is to increase the production of VLCFA into plants. Although the lipid and fatty acid content of seed oil can be modified by the traditional methods of plant breeding, the advent of recombinant DNA technology has allowed for easier manipulation of the oil content of a plant.

In order to increase or alter the levels of compounds such as seed oils in plants, nucleic acid sequences and proteins regulating lipid and fatty acid metabolism must be identified. In yeast, identification of the dehydratase of the elongase complex remained elusive until the recent identification of PHS1 as encoding this activity. The phs1 mutant was also characterized as a cell cycle mutant defective in G2/M phase. The biochemical function of Phs1 p as an hydroxyacyl-CoA dehydratase was provided by in vitro activity of recombinant protein and reconstitution of the elongase complex in proteoliposomes (Denic & Weissman, 2007). However, effects of the surexpression of this gene in vivo are unknown.

The role of the Arabidopsis PASTICCINO2 (PAS2) gene in regulation of the cellular cycle has been known for a while. Mutations in PAS2 gene lead to strong developmental defects mainly associated with ectopic cell division (Bellec et al., 2002; Faure et al., 1998; Haberer et al., 2002). This gene shares a significant similarity with the yeast dehydratase PHS1 (Bellec et al., 2002). Reponses to hormones like auxin and cytokinins that are essential for cell cycle progression and cell differentiation were also altered in pas2 mutant (Harrar et al., 2003). Finally, PAS2 was demonstrated to be able to interact with phosphorylated Cyclin dependent kinase and subsequently to prevent its dephosphorylation by CDC25-like phosphatase(s), preventing premature entry in mitosis (Da Costa et al., 2006).

### SUMMARY

The recent advances in plant molecular biology have made possible genetic engineering of most crop species. The technology has been applied to improving biosynthesis of VLCFAs in plant cells. Here, inventors showed for the first time that a recombinant plant cell expressing an heterologous gene encoding for an hydroxyacyl-CoA dehydratase is useful for the production of VLCFA.

In particular, inventors showed that PAS2 gene from Arabidopsis is associated with lipid biosynthesis and homeostasis. Indeed, PAS2 was found to be associated with ER and to physically interact with the reductase CER10, which was consistent with a role of dehydratase in the Arabidopsis microsomal elongase complex. An overexpression of the PAS2 gene leads to an increased production of VLCFA in recombinant plant cells. In the present application, a new method for the production of VLCFA into a plant cell is provided, comprising culturing a recombinant plant cell in an appropriate medium, wherein said plant cell comprises an heterologous gene encoding for an hydroxyacyl-CoA dehydratase, such as PAS2 from Arabidopsis thaliana.

### DETAILED DESCRIPTION

The invention is related to a method for the production of VLCFA into a plant cell, comprising culturing a recombinant plant cell in an appropriate medium, wherein said plant cell is transformed with an heterologous gene encoding for an hydroxyacyl coA dehydratase. As used herein, the following terms may be used for interpretation of the claims and specification. According to the invention, the term “VLCFA” refers to very long chain fatty acids, that are composed of 20 or more carbons in length (i.e. >C18). The term “plant cell” designates an isolated cell obtained from a plant by classical methods known by the man skilled in the art, such as a cell from any organ of a plant (seeds, leaves, roots, flowers) or cells that form in vitro grown plant cell cultures. The term “recombinant plant cell” designates a cell having been transformed with exogenous DNA, and having integrated this DNA.

The term “transformation” refers to the introduction of new genes or extra copies of existing genes into a plant cell. The acquired genes may be incorporated into chromosomal DNA or introduced as extra-chromosomal elements. As an example, for plant cells, a method for transferring DNA into a host organism is inoculation or infiltration of plant cells (from in vitro culture), of explants (like hypocotyls, roots) or of organs (like leaves or flowers) with Agrobacterium tumefaciens or Agrobacterium rizhogenes. Another method is the direct introduction of DNA (like electroporation or PEG mediated transfection) into plant protoplasts.

The term “culturing” includes maintaining and/or growing a living plant cell such that it can perform its intended function, i.e the production of fatty acids. A plant cell may be cultured in liquid media, in solid media, semi-solid media or in soil. An “appropriate medium” designates a medium (e.g., a sterile, liquid media) comprising nutrients essential or beneficial to the maintenance and/or growth of the cell such as carbon sources or carbon substrate, for example carbohydrate, hydrocarbons, oils, fats, fatty acids, organic acids, and alcohol's; nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids, vitamins, growth promoters, and the like.

The terms “encoding” or “coding” refer to the process by which a polynucleotide, through the mechanisms of transcription and translation, produces an amino-acid sequence. This process is allowed by the genetic code, which is the relation between the sequence of bases in DNA and the sequence of amino-acids in proteins. One major feature of the genetic code is to be degenerate, meaning that one amino-acid can be coded by more than one triplet of bases (one “codon”). The direct consequence is that the same amino-acid sequence can be encoded by different polynucleotides. It is well known from the man skilled in the art that the use of codons can vary according to the organisms. Among the codons coding for the same amino-acid, some can be used preferentially by a given microorganism. It can thus be of interest to design a polynucleotide adapted to the codon usage of a particular microorganism in order to optimize the expression of the corresponding protein in this organism.

The terms “enzyme activity” and “enzymatic activity” are used interchangeably and refer to the ability of an enzyme to catalyse a specific chemical reaction. The term “hydroxyacyl-CoA dehydratase” refers to a polypeptide responsible for an enzyme activity that catalyzes the “third step” of the VLCFA elongation, i.e. the dehydration of a 3-hydroxy-acyl-CoA to an enoyl-CoA. Such an enzyme activity of 3-hydroxy acyl-CoA dehydration was described in plants in (Lessire et al., 1999). Methods to measure this enzyme activity were provided in the same reference and in the recent work of (Kihara et al., 2008).

Inventors showed that this step of dehydration is a limiting step in the full processes of elongation. Therefore, increasing the amount or activity of this specific enzyme, among the four enzymes involved in the VLC fatty acids elongation, lead to a dramatic increase of production of VLCFA. In a particular embodiment of the invention, the heterologous gene is a gene sharing homology with the PAS2 gene from Arabidopsis, or a gene encoding for a protein sharing homology with the protein PAS2, such can be determined by the man skilled in the art. A protein sharing homology with the protein PAS2 may be obtained from plants or may be a variant or a functional fragment of a natural protein originated from plants.

The term “variant or functional fragment of a natural protein” means that the amino-acid sequence of the polypeptide may not be strictly limited to the sequence observed in nature, but may contain additional amino-acids. The term “a fragment” means that the sequence of the polypeptide may include less amino-acid than the original sequence but still enough amino-acids to confer hydroxyacyl CoA dehydratase activity. It is well known in the art that a polypeptide can be modified by substitution, insertion, deletion and/or addition of one or more amino-acids while retaining its enzymatic activity. For example, substitution of one amino-acid at a given position by a chemically equivalent amino-acid that does not affect the functional properties of a protein, are common. For the purpose of the present invention, substitutions are defined as exchanges within one of the following groups:
Small aliphatic, non-polar or slightly polar residues: Ala, Ser, Thr, Pro, Gly
Polar, negatively charged residues and their amides: Asp, Asn, Glu, Gln
Polar, positively charged residues: His, Arg, Lys
Large aliphatic, non-polar residues: Met, Leu, Ile, Val, Cys
Large aromatic residues: Phe, Tyr, Trp.
   Thus, changes that result in the substitution of one negatively charged residue for another (such as glutamic acid for aspartic acid) or one positively charged residue for another (such as lysine for arginine) can be expected to produce a functionally equivalent product.

The positions where the amino-acids are modified and the number of amino-acids subject to modification in the amino-acid sequence are not particularly limited. The man skilled in the art is able to recognize the modifications that can be introduced without, affecting the activity of the protein. For example, modifications in the N- or C-terminal portion of a protein may be expected not to alter the activity of a protein under certain circumstances.

The term “variant” refers to polypeptides submitted to modifications such as defined above while still retaining the original enzymatic activity. According to the invention, the polypeptide having an hydroacyl-CoA dehydratase enzymatic activity may comprise a sequence having at least 30% of homology with the sequence of PAS2, preferentially at least 50% of homology, and more preferentially at least 70% of homology.

Methods for the determination of the percentage of homology between two protein sequences are known from the man skilled in the art. For example, it can be made after alignment of the sequences by using the software CLUSTALW available on the website http://www.ebi.ac.uk/clustalw/ with the default parameters indicated on the website. From the alignment, calculation of the percentage of identity can be made easily by recording the number of identical residues at the same position compared to the total number of residues. Alternatively, automatic calculation can be made by using for example the BLAST programs available on the website http://www.ncbi.nlm.nih.gov/BLAST/with the default parameters indicated on the website.

Preferred genes encoding proteins according to the invention are selected among genes presented in FIG. 1, i.e. genes from Vitis vinifera (encoding CAN64341.1 hypothetical protein), Oryza sativa (CAD39891.2, EAY72548.1 hypothetical protein Osl_{—}000395, EAZ30025.1 hypothetical protein OsJ_{—}013508 and BAD61107.1 tyrosine phosphatase-like), Brassica rapa (AAZ66946.1), Hyacinthus orientalis (AAT08740.1 protein tyrosine phosphatase), Ostreacoccus lucimarinus (XP_{—}001420997.1 predicted protein and XP_{—}001422898.1 predicted protein), Chlamydomonas reinhardtii (EDP01055.1 predicted protein), and also from Brassica napus, Raphanus sativus, Brassica oleracea. In a specific embodiment of the invention, the heterologous gene is the gene PAS2 from Arabidopsis thaliana, registered in UniGene databank under number NP_{—}196610.2, also known as F12B17.170; F12B17_{—}170; PASTICCINO 2; PEP; and PEPINO. In another specific embodiment of the invention, the heterologous gene is the PHS1 gene from Saccharomyces cerevisiae, registered in gene databanks under number NP_{—}012438.1.

In another embodiment of the invention, the heterologous gene is from the same species than the species of the host plant cell. In a preferred embodiment of the invention, the heterologous gene is under the control of a promoter allowing the expression of said gene in the host plant cell. Preferentially, said promoter is a seed-specific promoter. This term “seed-specific promoter” means that a gene expressed under the control of the promoter is predominantly expressed in plant seeds with no substantial expression, typically less than 5% of the overall expression level, in other plant tissues.

Seed-specific plant promoters are known to those of ordinary skill in the art and are identified and characterized using seed-specific mRNA libraries and expression profiling techniques. Seed-specific promoters include the napin-gene promoter from rapeseed, the USP-promoter from Vicia faba, the oleosin-promoter from Arabidopsis, the phaseolin-promoter from Phaseolus vulgaris, the Bce4-promoter from Brassica or the legumin B4 promoter as well as promoters conferring seed specific expression in monocot plants like maize, barley, wheat, rye, rice etc. In a specific embodiment of the invention, the promoter is the promoter of the gene Napin from Arabidopsis (Accession number: At4g27150); for reference see (Guerche et al., 1990).

In another embodiment of the invention, the promoter used in the invention is an inducible promoter. Chemically inducible promoters are especially suitable if gene expression is desired in a time specific manner. Examples for such promoters are a salicylic acid inducible promoter, a tetracycline inducible promoter and an ethanol inducible promoter. Promoters responding to biotic or abiotic stress conditions are also suitable promoters such as the pathogen inducible PRPI-gene promoter, the heat inducible hsp80-promoter from tomato, cold inducible alpha-amylase promoter from potato or the wound-inducible pinII-promoter.

In a specific embodiment of the invention, the promoter may be chosen in a way to obtain gene expression in a time specific manner; for example, the man skilled in the art might chose between the following list of Arabidopsis promoters:

| | | Expression | |
|---|---|---|---|
| Gene name | Accession | pattern | references |
| ABI3 | At3g24650 | early | 1 |
| At2S3 | At4g27160 | late | 2 |
| FUSCA3 | At3g26790 | late | 3 |
| OLEOSIN 1 | At4g25140 | late | 4, 5 |
| OLEOSIN 2 | At5g40420 | late | 4, 5 |
| OLEOSIN 3 | At5g50770 | late | 4, 5 |
| OLEOSIN 4 | At3g27660 | late | 4, 5 |

References:
1, Despres (2001);
2, Guerche (1990);
3, Luerssen (1998);
4, Plant, (1994);
5, Siloto (2006)

All these promoters could be used for expressing a gene encoding for an hydroxyacyl-CoA dehydratase in the seed.

In a specific embodiment of the invention, at least one another gene involved in the VLCFA biosynthesis is introduced into the plant cell. In particular, this gene encodes for another or several enzyme(s) belonging to the elongase complex. Preferentially, said at least one gene is encoding for an enzyme selected from the following list: a fatty acid elongase, a reductase and combinations thereof. In a particular way to realize the invention, the recombinant plant cell is a cell from the seed.

The invention is also related to a method for the production of VLCFA into plants, comprising culturing a plant comprising at least one cell transformed with an heterologous gene encoding for an hydroxyacyl-CoA dehydratase. In a specific embodiment of the invention, the totality of the cells from the plant was transformed with the heterologous gene, and the plant is said “transformed plant” or “transgenic plant”. The term “production of VLCFA” designates the fact that the plant biosynthesizes a detectable amount of VLCFA. Quantities that might be obtained are shown in the examples, in particular in FIGS. 2 and 3, wherein VLCFA productions were analysed and compared from seeds from different genotypes.

The transformed plant may be chosen among Arabidopsis thaliana, Brassica napus, Brassica juncea, Helianthus anuus, and all other plants that may be determined as useful by the man skilled in the art. In particular, the invention could be applied to other plants including rapeseed, canola, linseed, soybean, sunflower, maize, oat, rye, barley, wheat, rice, pepper, tagetes, cotton, oil palm, coconut palm, flax, castor, and peanut. Preferentially, the method according to the invention further comprises a step of extraction of the VLCFA from the cell plant or from the plant. Techniques for extraction of fatty acids from plants are well known by the man skilled in the art, and comprise in particular gas chromatography; see for reference Baud et al. (2002).

This invention is also related to a method for producing vegetable oil, comprising the following steps:
culturing a plant comprising at least one cell transformed with an heterologous gene encoding for an hydroxyacyl-CoA dehydratase such as defined previously, and
extracting the oil from the plant.
   Said vegetable oil is advantageously enriched in VLCFA. Finally, the invention is also related to a method for identifying plants having a high potential of VLCFA biosynthesis, wherein said plants are selected on their level of expression or level of activity of hydroxyacyl-CoA dehydratase.

### BRIEF DESCRIPTION OF THE DRAWINGS

FIG. 1: Phylogenetic analysis of PAS2 homologs in plants. PAS2 protein homologs were identified by BLASTp from plant protein database (BCBI) and the resulting sequences were aligned using CLUSTALw. Graphical representation of sequence identities is presented as phylogenetic rooted tree.

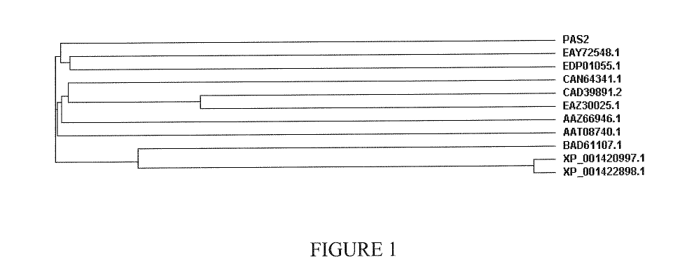

FIG. 2: Acyl-CoA dehydratase is an essential and limiting activity.

(A) Total fatty acid levels in the roots of pas2-1 and PHS1 expressing plants compared to wild type.
(B) Seed dry weight of pas2-1 and PHS1 expressing plants compared to wild type.
(C) Total fatty acid levels in pas2-1 and PHS1 expressing plants compared to wild type.
Dry weight and fatty acid values are the average of three samples±sd.

FIG. 3: VLCFA production in plant seeds transformed with PAS2, PHS1, Col0 or deleted of the PAS2 gene. VLCFA levels are presented as values of each class (assessed by the length of the acyl chain) relative to the total amount of VLCFA (expressed in % of absolute values in nmol/mg fresh weight).

### EXAMPLES

### Example 1

### Arabidopsis Cells Expressing an Heterologous Gene PHS1 from Yeast Produce Higher Levels of VLCFA

The orthologous yeast PHS1 gene was introduced into Arabidopsis plant cell to monitor the effect of increasing dehydratase activity on VLCFA levels and on plant development. PHS1 was cloned under the control of the ubiquitous 35S promoter. Several independent lines expressing PHS1 showed clear growth retardation associated with abnormal leaf development:
Leaves from transgenic lines were smaller and more crinkled than that of control plants.
They also showed altered shapes with pronounced serration and often an asymmetric development of the leaf blade leading to a sickled shape.
Epidermal cells from PHS1 expressing transgenic leaves were characterized by a large heterogeneity in cell sizes and shapes.
the surface of PHS1-expressing leaf epidermal cells was decorated with wax crystals suggesting an increase in cuticular waxes in contrast to wild type (FIG. 5C).
Flower development was also modified by PHS1 expression with for instance misshapen and unfused carpels.
Detailed analysis of cell surface of unfused carpel showed high accumulation of cuticular waxes.
   Extraction and analyses of fatty acid methyl esters by gas chromatography were performed as described previously in (Baud et al., 2002) and modified according to (Li et al., 2006).

For lipid extraction, 20 seeds were ground in a glass reaction tube in 250 μl of chloroform/methanol/acetic acid/water (10:10:1:1, v/v/v/v) and incubated at −20 C overnight. Then, 92 μl of chloroform/methanol/water (5:5:1, v/v/v) and 125 μl of Hajra solution (2 M KCl and 0.2 M H3PO4) were added. After shaking and centrifugation the lower phase, which contains lipids, was transferred to a new glass tube and stored at −20 C. For total fatty, acid quantity and composition analyses by gas chromatography of the corresponding fatty acyl methyl esters, extracted lipids were incubated in 1 ml of methanol/sulphuric acid (100:2.5, v/v) at 80° C. for 30 min after addition of 17:0 fatty acid as an internal standard. Fatty acyl methyl esters were then extracted into 450 μl of hexane following the addition of 1.5 ml of water. After vigorous shaking and centrifugation, 1 μl of the upper organic phase was analysed by gas chromatography. Fatty acid methyl esters were, separated by GC on a 15-m×0.53-mm Carbowax column (Alltech, France) and quantified using a flame ionisation detector. The gas chromatograph was programmed for an initial temperature of 160 C for 1 min followed by a 40 C/min ramp to 190 C and a secondary ramp of 4 C/min to 230 C; this final temperature was maintained for 2 min.

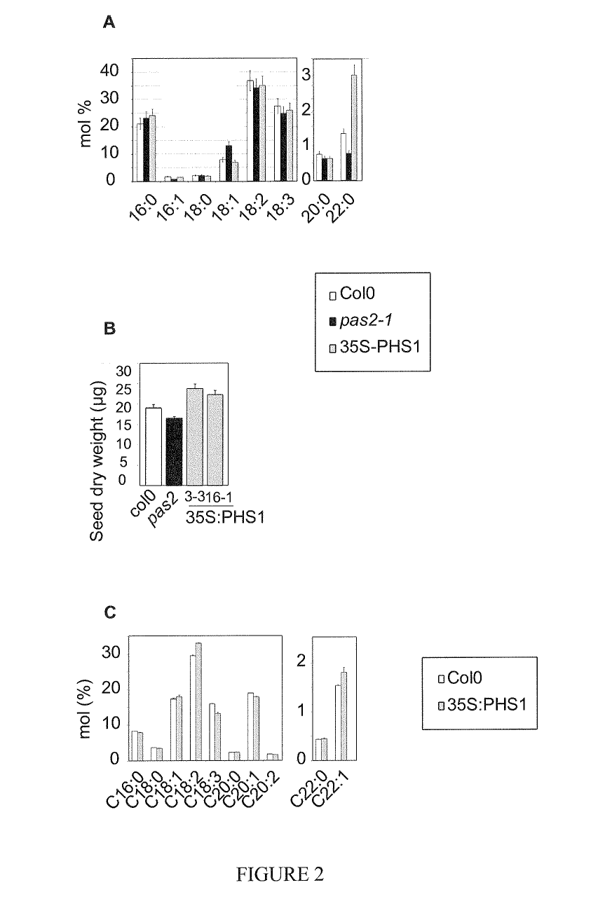

Analysis of fatty acid content of roots of young seedlings showed that ectopic expression of PHS1 modified VLCFA content. Indeed, the 35S:PHS1 seedlings showed significative changes in the relative distribution of VLCFAs with higher levels of 22:0 compared to wild type (FIG. 2A). Since VLCFAs are also normally found in mature seeds, we investigated the effect of PHS1 expression on seed size and total fatty acid levels. Expression of PHS1 led to slightly larger seeds while pas2 mutant showed smaller seeds compared to wild type (FIG. 2B). Similarly to that observed with seedlings, PHS1 expressing seeds showed an increase in VLCFAs mostly 22:1 (FIG. 2C).

In roots, fatty acid analysis showed a similar effect of Phs1 in roots compared to seeds. Phs1 expressing plants do not show any increase of c20 fatty acids (levels are actually decreased by 20%). The levels of longer fatty acids like 22:0 and 24:0 were increased respectively by 54 and 44%.

In conclusion, VLCFA dehydratase, is not only an essential enzyme for plant growth and development but it is also a limiting step for VLCFA synthesis since an increased dehydratase expression resulted in enhanced levels of VLCFAs in both vegetative and seed tissues.

### Example 2

### Ectopic PHS1 and PAS2 Expression in Mature Seeds Lead to an Increase in VLCFAs Mostly C22:0 and C22:1

For seed fatty acid analysis, 20 mature seeds were ground in clean glass tube with 1 ml of methanol/toluene/H2504 (1:0.3 v:v plus 0.25% H2SO4 v/v). Then, samples are incubated at 80° C. checking after 1 or 2 minutes for any leak. After 90 minutes, tubes are removed from heat, and fatty acyl methyl esters were then extracted into 450 μl of hexane following the addition of 1.5 ml of water. After vigorous shaking and centrifugation, 1 μl of the upper organic phase was analysed by gas chromatography. Fatty acid methyl esters were separated by GC. To estimate the total fatty acids, 10 μg of C17:0 per mL of sulfuric methanol toluene were added.

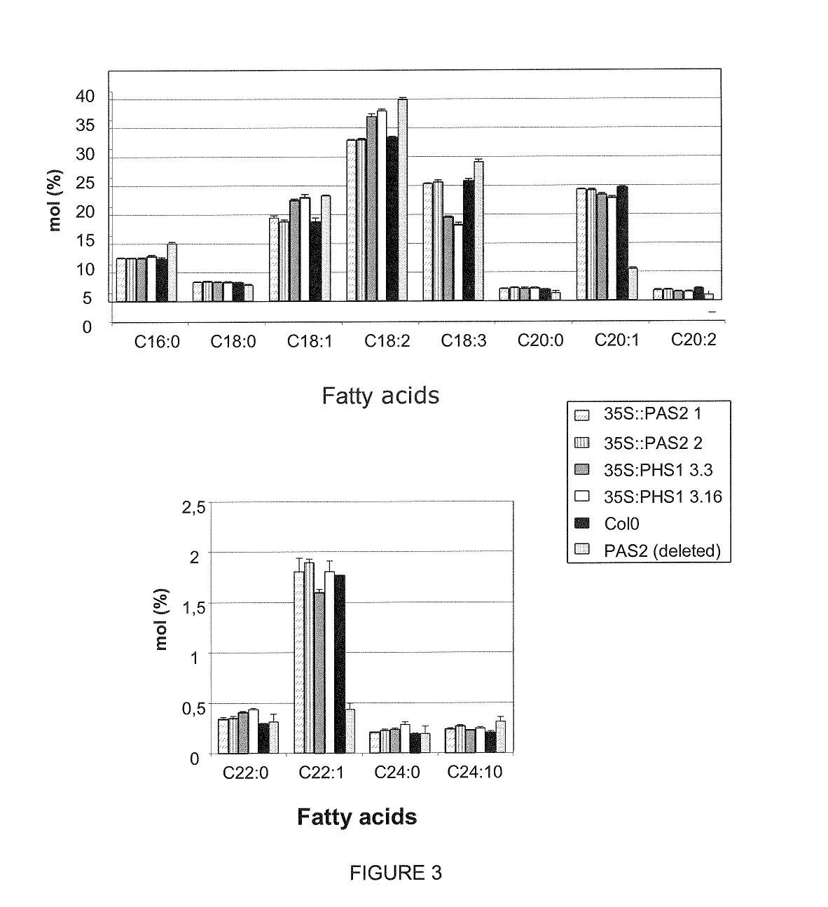

The VLCFAs seed composition were analyzed for two independent lines expressing either PHS1 (lines 3.3 and 3.16) or PAS2 (lines 1 and 2) under the control of the 35S promoter and compared with wild type (accession Columbia-0, Col0). Plant cells “PAS2” designates mutant cells whose PAS2 gene is deleted. Results are presented in FIG. 3.

### REFERENCES IN ORDER OF THE CITATION IN THE TEXT

Denic V, Weissman J S (2007) A molecular caliper mechanism for determining very long-chain fatty acid length. Cell 130: 663-77.
Bellec Y, Harrar Y, Butaeye C, Darnet S, Bellini C, et al. (2002) Pasticcino2 is a protein tyrosine phosphatase-like involved in cell proliferation and differentiation in Arabidopsis. Plant J 32: 713-22.
Faure J D, Vittorioso P, Santoni V, Fraisier V, Prinsen E, et al. (1998) The PASTICCINO genes of Arabidopsis thaliana are involved in the control of cell division and differentiation. Development 125: 909-918.
Haberer G, Erschadi S, Torres-Ruiz R A (2002) The Arabidopsis gene PEPINO/PASTICCINO2 is required for proliferation control of meristematic and non-meristematic cells and encodes a putative anti-phosphatase. Dev Genes Evol 212: 542-50.
Harrar Y, Bellec Y, Bellini C, Faure J D (2003) Hormonal control of cell proliferation requires PASTICCINO genes. Plant Physiol 132: 1217-27.
Da Costa M, Bach L, Landrieu I, Bellec Y, Catrice O, et al. (2006) Arabidopsis PASTICCINO2 Is an Antiphosphatase Involved in Regulation of Cyclin-Dependent Kinase A. Plant Cell 18: 1426-37.
Lessire, R., S. Chevalier, et al. (1999). “Study of the 3-hydroxy eicosanoyl-coenzyme A dehydratase and (E)-2,3 enoyl-coenzyme A reductase involved in acyl-coenzyme A elongation in etiolated leek seedlings.” Plant Physiol 119(3): 1009-16.
Kihara, A., H. Sakuraba, et al. (2008). “Membrane topology and essential amino acid residues of Phs1, a 3-hydroxyacyl-CoA dehydratase involved in very long-chain fatty acid elongation.” J Biol Chem 283(17): 11199-209.
Guerche P, Tire C, De Sa F G, De Clercq A, Van Montagu M, Krebbers E. (1990). “Differential Expression of the Arabidopsis 2S Albumin Genes and the Effect of Increasing Gene Family Size.” Plant Cell. 1990 May; 2(5):469-478.
Baud, S., J. P. Boutin, et al. (2002). “An integrated overview of seed development in Arabidopsis thaliana ecotype Ws.” Plant Physiol. Biochem. 40: 151-160.
Li, Y., F. Beisson, et al. (2006). “Oil content of Arabidopsis seeds: the influence of seed anatomy, light and plant-to-plant variation.” Phytochemistry 67(9): 904-15.

1. A method for the production of VLCFA into a plant cell, comprising culturing a recombinant plant cell in an appropriate medium, wherein said plant cell is transformed with an heterologous gene encoding for an hydroxyacyl-CoA dehydratase.

2. The method according to claim 1, wherein said heterologous gene is selected among genes from Saccharomyces cerevisiae, Arabidopsis thaliana, Vitis vinifera, Oryza sativa, Brassica rapa, Hyacinthus orientalis, Ostreacoccus lucimarinus, Chlamydomonas reinhardtii, Brassica napus, Raphanus sativus, and Brassica oleracea.

3. The method according to claim 1, wherein the heterologous gene is the gene PAS2 from Arabidopsis thaliana.

4. The method according to claim 1, wherein the heterologous gene is the PHS1 gene from Saccharomyces cerevisiae.

5. The method according to claim 1, wherein the heterologous gene is under the control of a promoter.

6. The method according to claim 5, wherein the heterologous gene is under the control of a seed-specific promoter.

7. The method according to claim 5, wherein the heterologous gene is under the control of an inductible promoter.

8. The method according to claim 1, wherein at least one other gene involved in the VLCFA biosynthesis is introduced into the cell.

9. The method according to claim 9, wherein said at least one gene is encoding for an enzyme selected from the group consisting in: a fatty acid elongase, a reductase, and combinations thereof.

10. The method according to claim 1, wherein the plant cell is a seed cell.

11. A method for the production of VLCFA into plants, comprising culturing a plant comprising at least one cell transformed with an heterologous gene encoding for an hydroxyacyl-CoA dehydratase.

12. The method according to claim 11, wherein the plant is chosen among Arabidopsis thaliana, Brassica napus, Brassica juncea and Helianthus anuus.

13. The method according to claim 1, comprising a step of extraction of the VLCFA from the plant cell or from the plant.

14. A method for producing vegetable oil, comprising:
culturing a plant comprising at least one cell transformed with an heterologous gene encoding for an hydroxyacyl-CoA dehydratase, and
extracting the oil from the transformed plant.

15. A method for identifying plants having a high potential of VLCFA biosynthesis, wherein plants are selected on their level of expression or level of activity of hydroxyacyl-CoA dehydratase.

16. The method according to claim 5, wherein the heterologous gene is under the control of a seed-specific inductible promoter.

17. The method according to claim 11, comprising a step of extraction of the VLCFA from the plant.
